(19) United States
(12) Patent Application Publication
(10) Pub. No.: US 2021/0046161 A1
(43) Pub. Date: Feb. 18, 2021

(54) SKIN OINTMENT FOR TREATING LABIA MINORA SKIN

(71) Applicant: BNV BIOLAB. CO., LTD., Seoul (KR)

(72) Inventor: Sung Hun Suh, Seoul (KR)

(73) Assignee: BNV BIOLAB. CO., LTD., Seoul (KR)

(21) Appl. No.: 17/051,165

(22) PCT Filed: May 21, 2019
(86) PCT No.: PCT/KR2019/006065

§ 371 Date: Oct. 27, 2020

(30) Foreign Application Priority Data
Jul. 25, 2018 (KR) 10-2018-0086840

(51) Int. Cl. A61K 38/20; A61K 38/18; A61K 9/06; A61K 47/26; A61K 47/12; A61K 38/19; A61K 47/36; A61P 15/02; A61K 47/10; A61P 17/02; A61K 47/44; A61K 47/06

(52) U.S. Cl. CPC A61K 38/2053; A61K 38/1866; A61K 38/1825; A61K 9/06; A61K 47/26; A61K 47/12; A61K 38/1858; A61K 38/204; A61K 38/1833; A61K 38/191; A61K 47/36; A61P 15/02; A61K 38/2046; A61K 47/10; A61P 17/02; A61K 38/193; A61K 47/44; A61K 47/06; A61K 38/1808

(57) ABSTRACT

According to a skin ointment for treating labia minora skin according to the present disclosure, the skin ointment includes multi-growth factors as active ingredients, and is periodically applied to the vulva of a user so that the growth factors induce the regeneration of skin cells of the user, thereby increasing the effect of treating vulvar vestibulitis or wounds due to labia minora surgery, and addressing the fundamental causes thereof.

### TECHNICAL FIELD

The present disclosure relates to a skin ointment, and more particularly to a skin ointment for treating labia minora skin.

### BACKGROUND ART

The labia minora refers to the thin skin inside the labia majora in outer reproductive organs among the female reproductive organs, and there are cases where, when the labia minora is too large or asymmetric, it causes discomfort in daily life, and the growth of bacteria or fungi in summer or in a high-temperature environment is higher than that in the general population, and thus may increase the incidence of vaginitis, vulvar vestibulitis, or the like. In order to alleviate this discomfort in life, there are cases where labia minora surgery is performed. In addition to surgery for therapeutic purposes, recently, there are cases where labia minora surgery for cosmetic purposes is performed.

[symbol]

FIG. 1 is a view for explaining the surgical trend of labial hypertrophy. Through FIG. 1, it can be confirmed that the proportion of patients who have a reoperation after laser treatment on the labia minora is quite high. From the fact that the rate of reoperation after surgery to make the shape of the labia minora symmetrical or reduce the size thereof is high, it can be seen that there are cases where satisfaction with labia minora surgery is low, and care after labia minora surgery is also important. When care is not taken properly after labia minora surgery, a labia minora skin disease may be caused, or dryness may occur around the labia minora, leading to discomfort. Thus, for care and quick recovery, an ointment is actually prescribed to help the recovery of labia minora skin after labia minora surgery. In this case, as the ointment applied to the surgical site of the labia minora, an ointment mainly used in the treatment of vaginitis is used.

[symbol]

Vaginitis is a disease that can occur in women, and symptoms thereof include excessive vaginal discharge or bad odors from vaginal discharge, and the case of suffering from leukorrhea may also be considered a symptom of vaginitis. Vaginitis can be caused by bacterial infections that cause bacteria to proliferate as acidity in the vagina decreases due to a reduction in the number of lactic acid bacteria, which maintain an acidic environment in the vagina. In addition, vaginitis can be caused by non-infectious vaginitis, which occurs due to hormonal imbalance and malfunction of the immune system or metabolism. In modern society, immunity is often lowered due to stress, excessive diet, and the like, and thus the incidence of vaginitis in young women as well as older women tends to increase. In addition, as a female disease, vaginal dryness is caused by a decrease in moisture in the mucous membrane layer of the vagina and thinning of the vaginal wall, and as a therapeutic agent therefor, a hormonal ointment is also generally used. Vaginal dryness was a symptom that only appeared in female patients in their 40s or 50s or older until the early 2000s, but due to lowered immunity caused by excessive diet, the number of patients in their teens and 20s is increasing, and thus interest in the disease is increasing.

[symbol]

For recovery after labia minora surgery and the treatment of vaginitis, vaginal dryness, and the like as described above, most hospitals present hormonal ointments and the like as a solution, and in fact, ointments containing female hormones, steroids, and the like are prescribed. As cited references related to this, there are registered patent No. 10-0400411 (Title of Disclosure: Probiotics for Treating Vaginitis), and the like.

[symbol]

However, in the case of these hormonal ointments, there is a problem in that the effects are temporary or almost ineffective. In addition, when application of an ointment is stopped, symptoms may reappear. This is suspected as a symptom of addiction to an ointment, and thus not only patients but also doctors are reluctant to use products containing hormones or steroids. In addition, in the case of therapeutic agents containing female hormones, the biggest problem is that these increase the incidence of cancer in women, and in the case of young women, there are also increasing cases of side effects due to the occurrence of hormonal imbalances. Due to these problems, there is a need to develop an ointment that does not contain hormones and is highly effective in the treatment of symptoms.

### DESCRIPTION OF EMBODIMENTS

### Technical Problem

To address the above-described problems of existing methods proposed, the present disclosure provides a skin ointment for treating labia minora skin, which includes multi-growth factors as active ingredients, and is periodically applied to the vulva of a user so that the growth factors induce the regeneration of skin cells of the user, thereby increasing the effect of treating vulvar vestibulitis or wounds due to labia minora surgery, and addressing the fundamental causes thereof.

[symbol]

The present disclosure also provides a skin ointment for treating labia minora skin, which contains high concentrations of multi-growth factors and is prepared as an ointment formulation, and thus is easily applied to the vulva of a user, and which, by excluding sex hormones, environmental hormones, steroid hormones, and antibiotic ingredients, does not break the hormonal balance of the user and can be used safely by pregnant or breast-feeding women.

### Solution to Problem

According to an aspect of an embodiment, a skin ointment for treating labia minora skin

includes

multi-growth factors as active ingredients.

[symbol]

Preferably,

the skin ointment may be periodically applied to the vulva of a user, thereby treating vulvar vestibulitis or wounds due to labia minora surgery.

[symbol]

Preferably, the multi-growth factors may be

multi-growth factors including FGF, bGFG, GM-CSF, HGF, IGF-1, IL-6,7,8, KGF, Matrix proteins, PDGF-AA, TGF-B2, TGF-B3, EGF, VEGF, and TNF.

[symbol]

Preferably,

the multi-growth factors may be included at a concentration of 200,000 ppm to 400,000 ppm on a weight basis.

[symbol]

More preferably,

the multi-growth factors may be included at a concentration of 300,000 ppm on a weight basis.

[symbol]

More preferably,

the skin ointment may be prepared as an ointment formulation including purified water, glycerin, Simmondsia chinensis (jojoba) seed oil, xanthan gum, a hydroxyethyl acrylate/sodium acryloyldimethyl taurate copolymer, squalane, polysorbate 60, Rosmarinus officinalis (rosemary) leaf oil, lactic acid, chlorphenesin, and sorbitan isostearate.

[symbol]

Preferably,

the skin ointment may exclude female hormones, environmental hormones, steroid hormones, and antibiotic ingredients.

### Advantageous Effects of Disclosure

According to a skin ointment for treating labia minora skin according to the present disclosure, the skin ointment includes multi-growth factors as active ingredients, and is periodically applied to the vulva of a user so that the growth factors induce the regeneration of skin cells of the user, thereby increasing the effect of treating vulvar vestibulitis or wounds due to labia minora surgery, and addressing the fundamental causes thereof.

[symbol]

In addition, according to the present disclosure, the skin ointment includes high concentrations of multi-growth factors and is also prepared as an ointment formulation, and thus is easily applied to the vulva of a user, and which, by excluding sex hormones, environmental hormones, steroid hormones, and antibiotic ingredients, does not break the hormonal balance of the user and can be used safely by pregnant or breast-feeding women.

### BRIEF DESCRIPTION OF DRAWINGS

FIG. 1 is a view for explaining the surgical trend of labial hypertrophy.

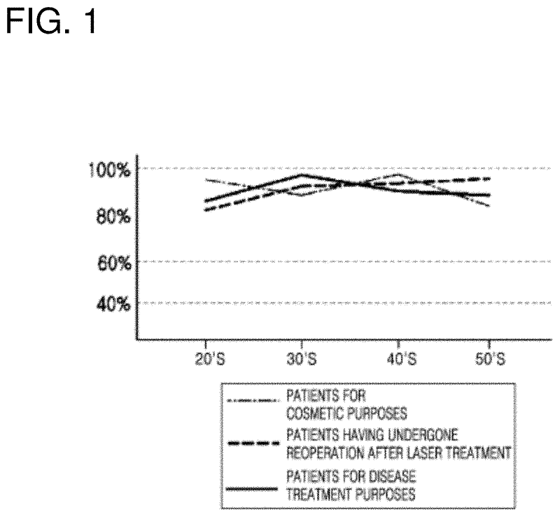

FIG. 2 is a view for explaining the efficacy of growth factors.

FIG. 3 is a view showing the ingredients of a skin ointment for treating labia minora skin according to an embodiment of the present disclosure.

FIG. 4 shows images showing an observed state of the degree of recovery through application of a skin ointment for treating labia minora skin according to an embodiment of the present disclosure to patients who have undergone labia minora surgery and clitoroplasty.

FIG. 5 shows images showing an observed state of the degree of recovery through application of a skin ointment for treating labia minora skin according to an embodiment of the present disclosure to patients who have undergone surgery for labial hypertrophy.

FIG. 6 shows images showing an observed state of the degree of recovery through application of a skin ointment for treating labia minora skin according to an embodiment of the present disclosure to patients diagnosed with vulvar vestibulitis and atrophic vaginitis.

### BEST MODE

Hereinafter, example embodiments will be described in detail with reference to the accompanying drawings in such a manner that the present disclosure may be easily carried out by one of ordinary skill in the art to which the present disclosure pertains. However, in detailed description of example embodiments of the present disclosure, detailed description of related functions or configurations will be omitted when it is deemed that they may unnecessarily obscure the essence of the present disclosure. In addition, components having similar functions and actions are denoted by like reference numerals throughout the drawings.

[symbol]

In addition, in the entire specification, when a part is referred to as being “connected” with another part, it includes not only a case where it is directly connected, but also a case where a part is indirectly connected with another part with another element disposed therebetween. In addition, “including” an element means that, unless otherwise specifically stated, other elements are not precluded and also may mean that other elements are further included.

[symbol]

A skin ointment for treating labia minora skin according to an embodiment of the present disclosure includes multi-growth factors as active ingredients. Previously, products used for vaginitis and the like have been used to treat labia minora skin, but the skin ointment for treating labia minora skin according to the present disclosure, which has a function of inducing regeneration by using growth factors, particularly multi-growth factors, has a distinct difference from existing ointment products that do not use growth factors. In particular, the present disclosure is meaningful in that the skin ointment is not a temporary pain relief product, but regenerates skin tissue and a vaginal mucosa layer, which addresses the fundamental causes of symptoms.

[symbol]

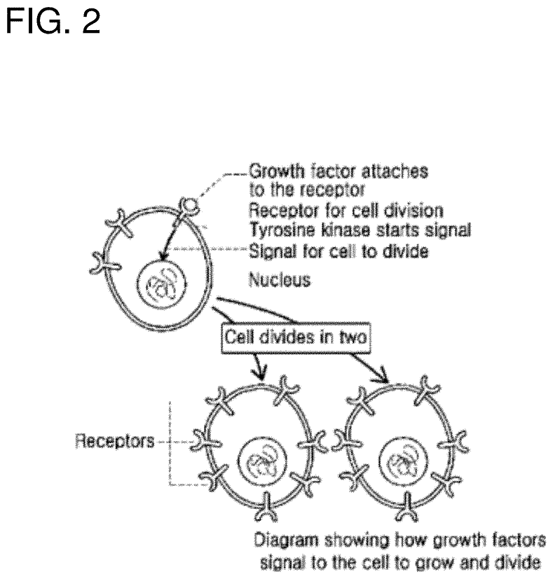

FIG. 2 is a view for explaining the efficacy of growth factors. As illustrated in FIG. 2, growth factors promote the growth, proliferation, and differentiation of cells by binding to the receptors of the cells, thereby enabling the natural regeneration cycle of the skin of a user to smoothly proceed. That is, growth factors are responsible for increasing the amounts of collagen and elastin in the skin and regenerating the skin, and since the labia minora skin of women also consists of collagen, the efficacy of growth factors in facilitating the regeneration cycle of cells may be obtained, and ultimately, fundamental causes may be addressed.

[symbol]

In the present disclosure, growth factors are absorbed into the labia minora skin of a user to induce the regeneration of skin cells of the user, and may be multi-growth factors including FGF, bGFG, GM-CSF, HGF, IGF-1, IL-6,7,8, KGF, Matrix proteins, PDGF-AA, TGF-B2, TGF-B3, EGF, VEGF, and TNF. In particular, the skin ointment for treating labia minora skin according to an embodiment of the present disclosure does not use EGF or FGF alone as a growth factor, but may contain multi-growth factors as listed above in the form of a multi-peptide factor protein.

[symbol]

Growth factors have different functions. For example, EGF is an epithelial growth factor that helps the growth of epithelial cells, and FGF is a growth factor that stimulates the production of fibroblasts, has a function of strengthening wound healing ability and promoting skin regeneration, and is involved in skin tissue reconstruction by secreting collagen. In addition, PDGF stimulates angiogenesis, removes wound tissue, and promotes healing, and KGF stimulates the division and differentiation of angiogenic cells, improves the function of monocytes and macrophages, and promotes the proliferation of epidermal cells. Since cells constituting the body do not consist of only single cells, the skin ointment for treating labia minora skin according to an embodiment of the present disclosure includes multi-growth factors, and thus may help the proliferation and regeneration of various cells using various growth factors, thereby obtaining a more rapid regeneration rate and effect.

[symbol]

For the treatment of labia minora skin, the skin ointment for treating labia minora skin according to an embodiment of the present disclosure including multi-growth factors may be applied to the skin of a corresponding site to enable the multi-growth factors to permeate into the skin of a user, thereby enhancing the effect of ameliorating and treating labia minora skin diseases. Growth factors are advantageous in terms of no immune rejection response and no side effects.

[symbol]

The skin ointment for treating labia minora skin according to an embodiment of the present disclosure may include multi-growth factors at a concentration of 200,000 ppm to 400,000 ppm on a weight basis, and more particularly, may include the multi-growth factors at a concentration of 300,000 ppm on a weight basis. That is, the multi-growth factors included in the skin ointment for treating labia minora skin according to an embodiment of the present disclosure is in the form of a conditioned medium of growth factor with 20% to 40% high-concentration, preferably a conditioned medium of growth factor with 30% high-concentration. In some embodiments, the conditioned medium of growth factor with 30% high-concentration may be a mixture of natural nutrients and moisturizing ingredients.

[symbol]

Conventional skin products including growth factors are mostly 1,000 ppm or less, and the concentration is not disclosed separately due to the low concentration. Thus, even a concentration of about 2,000 ppm may be distinguished from conventional skin products. The skin ointment for treating labia minora skin according to an embodiment of the present disclosure includes a high concentration of multi-growth factors of 200,000 ppm to 400,000 ppm, which is 10-20 times that of growth factors included in conventional skin products. Due to such a difference, the effect of the skin ointment on the amelioration and treatment of skin has a significant difference from that of low-concentration products.

[symbol]

In particular, the skin ointment for treating labia minora skin according to an embodiment of the present disclosure uses growth factor conditioned media to produce growth factors that are most optimized for the skin and tissue of the human body, thereby maximizing the effect of regenerating the skin tissues and cell membranes of the labia minora. In other words, when growth factors in the form of powder obtained by pulverizing a frozen powder form is added during product formulation, it is difficult to accurately specify the content thereof. In addition, since growth factors themselves are proteins, when these are frozen, the effect is remarkably reduced (80-90% or more) as compared to a protein form. In contrast, the skin ointment for treating labia minora skin according to an embodiment of the present disclosure uses growth factor conditioned media that maintain a protein form as it is, and thus, as compared to the case where growth factors in the form of frozen powder are used, a significantly excellent effect may be obtained.

[symbol]

In addition, the skin ointment for treating labia minora skin according to an embodiment of the present disclosure may exclude female hormones, environmental hormones, steroid hormones, and antibiotic ingredients, and thus can be safely used by pregnant or breast-feeding women. In some embodiments, the skin ointment may further include at least one of vitamins, amino acids, and hyaluronic acid.

[symbol]

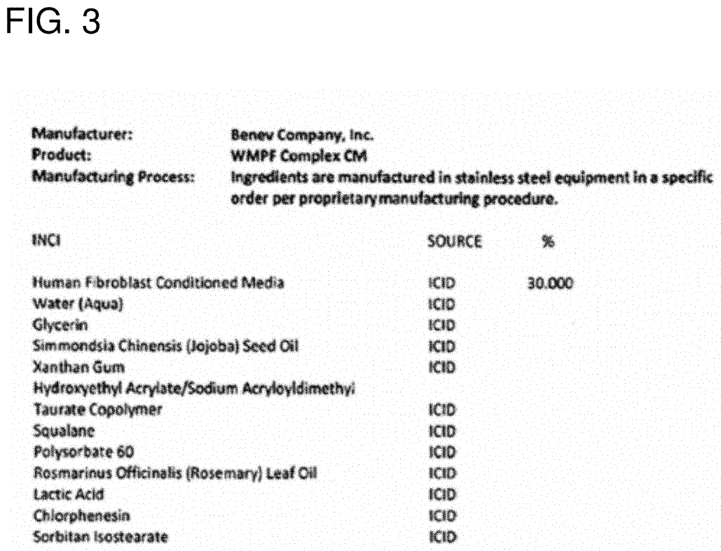

FIG. 3 is a view showing the ingredients of a skin ointment for treating labia minora skin according to an embodiment of the present disclosure. As illustrated in FIG. 3, the skin ointment for treating labia minora skin according to an embodiment of the present disclosure includes 30 wt % of multi-growth factors, and may be prepared as an ointment formulation including purified water, glycerin, Simmondsia chinensis (jojoba) seed oil, xanthan gum, a hydroxyethyl acrylate/sodium acryloyldimethyl taurate copolymer, squalane, polysorbate 60, Rosmarinus officinalis (rosemary) leaf oil, lactic acid, chlorphenesin, and sorbitan isostearate.

[symbol]

Most of conventional skin products including growth factors are in the form of ampoules or light creams. In particular, when growth factors are contained at a high concentration, it is difficult to prepare an ointment formulation, which is a semi-solid drug for external use. However, since the skin ointment for treating labia minora skin according to an embodiment of the present disclosure includes the ingredients as shown in FIG. 3, the skin ointment not only includes a high concentration of multi-growth factors of 300,000 ppm, but is also prepared as an ointment formulation, and thus is different from ampoule or cream types in terms of viscosity and tactility, which facilitates the application thereof to labia minora skin. In particular, it is not easy for a user to directly apply a light product to labia minora skin, and it is difficult for multi-growth factors to be sufficiently absorbed into the vulvar skin due to wearing of underwear, activities, and the like. Thus, the skin ointment for treating labia minora skin according to an embodiment of the present disclosure is prepared as a semi-solid skin ointment formulation including a high concentration of multi-growth factors, and thus is easily applied to labia minora skin and enables the high concentration of multi-growth factors to be sufficiently absorbed into the skin, and accordingly, has a significant effect on the treatment of vulvar vestibulitis or wounds due to labia minora surgery.

[symbol]

### EXAMPLE 1

Among from human fibroblasts (extracted from the foreskin) supplied from Tissue Bank at University of California, Ivine, healthy living fibroblasts were selected and cultured such that a constant growth factor concentration was maintained in all culture processes, thereby producing multi-growth factors in a conditioned media form. The produced multi-growth factors include FGF, bGFG, GM-CSF, HGF, IGF-1, IL-6,7,8, KGF, Matrix proteins, PDGF-AA, TGF-B2, TGF-B3, EGF, VEGF, and TNF. The multi-growth factors were mixed with purified water, glycerin, Simmondsia chinensis (jojoba) seed oil, xanthan gum, a hydroxyethyl acrylate/sodium acryloyldimethyl taurate copolymer, squalane, polysorbate 60, Rosmarinus officinalis (rosemary) leaf oil, lactic acid, chlorphenesin, and sorbitan isostearate, thereby completing the preparation of a skin ointment for treating labia minora skin as an ointment formulation, the skin ointment containing multi-growth factors at a concentration of 300,000 ppm on a weight basis. The prepared skin ointment was put in a 15 ml tube-shaped container.

[symbol]

### Experimental Example 1

The prepared skin ointment for treating labia minora skin, which was contained in a 15 ml tube-shaped container, was provided to women who had undergone labia minora surgery and clitoroplasty, and while the skin ointment was periodically applied to the vulva once a day, progression after surgery was observed for two weeks.

[symbol]

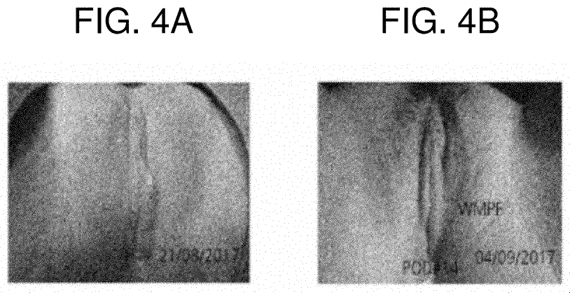

FIG. 4 shows images showing an observed state of the degree of recovery through application of a skin ointment for treating labia minora skin according to an embodiment of the present disclosure to patients who have undergone labia minora surgery and clitoroplasty, wherein (a) is an image before surgery, and (b) is an image after two weeks from surgery. As a result of periodically applying the skin ointment for treating labia minora skin according to the present disclosure once a day to the right side of the labia minora, which is a surgical site, it can be confirmed that a significant recovery was shown within two weeks.

[symbol]

### Experimental Example 2

The prepared skin ointment for treating labia minora skin, which was contained in a 15 ml tube-shaped container, was provided to women who had undergone surgery for labial hypertrophy, and while the skin ointment was periodically applied to the vulva once a day, progression after surgery was observed for 20 days.

[symbol]

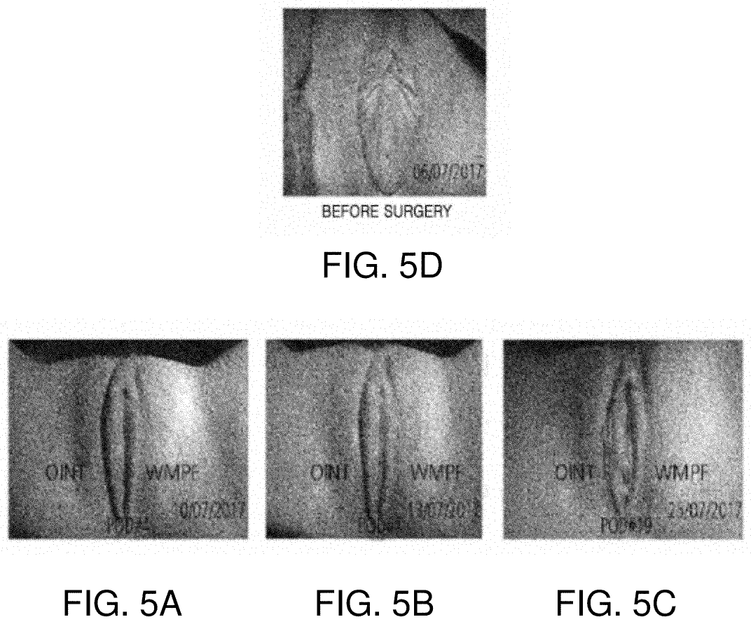

FIG. 5 shows images showing an observed state of the degree of recovery through application of a skin ointment for treating labia minora skin according to an embodiment of the present disclosure to patients who have undergone surgery for labial hypertrophy. The image on the top of FIG. 5 is an image before surgery, and among the images on the bottom of FIG. 5, (a) is an image after 4 days from surgery, (b) is an image after one week from surgery, and (c) is an image after 19 days from surgery. In (a), (b), and (c) of FIG. 5, the left side of the labia minora shows recovery through application of a hormone-containing ointment, which is generally prescribed after labia minora surgery (represented by “OINT” in the images), and the right side of the labia minora shows recovery through application of the skin ointment for treating labia minora skin according to an embodiment of the present disclosure (represented by “WMPF” in the images). As can be seen from (a), (b), and (c) of FIG. 5, skin regeneration of a site to which the skin ointment for treating labia minora skin according to an embodiment of the present disclosure had been applied was more rapid than that of a site to which a general ointment had been applied, and recovery in terms of the shape thereof also proceeded more smoothly. As such, the skin ointment for treating labia minora skin according to an embodiment of the present disclosure has a significant effect on the treatment of wounds due to labia minora surgery, as compared to existing general ointments, and also helps to recover the shape of the labia minora, thereby improving satisfaction with labia minora surgery.

[symbol]

### Experimental Example 3

The prepared skin ointment for treating labia minora skin, which was contained in a 15 ml tube-shaped container, was provided to women diagnosed with vulvar vestibulitis and atrophic vaginitis, and while the skin ointment was periodically applied to the vulva once a day, progression was observed for 48 days.

[symbol]

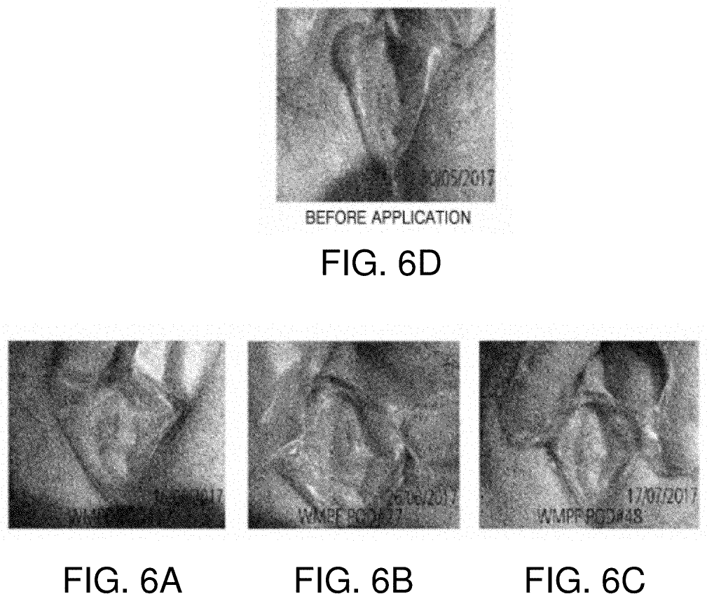

FIG. 6 shows images showing an observed state of the degree of recovery through application of a skin ointment for treating labia minora skin according to an embodiment of the present disclosure to patients diagnosed with vulvar vestibulitis and atrophic vaginitis. The image on the top of FIG. 6 is an image before the skin ointment for treating labia minora skin according to an embodiment of the present disclosure was applied, showing the vulva of a patient who has felt great discomfort in daily life for 10 years due to urination symptoms such as vulvar discomfort, urinary pain, frequent urination, nocturia, and urinary urgency, along with vulvar pain, dyspareunia, pelvic pain, lower abdominal pain, and back pain. The above patient was diagnosed with atrophic vaginitis and vulvar vestibulitis, and even after hormone therapy about three times, no therapeutic effect was observed. The degree of recovery of the vulva after the skin ointment for treating labia minora skin according to an embodiment of the present disclosure was periodically applied to the patient once a day was observed.

[symbol]

Among the images on the bottom of FIG. 6, (a) is an image after 17 days from application of the skin ointment of the present disclosure, (b) is an image after 27 days from application of the skin ointment of the present disclosure, and (c) is an image after 48 days from application of the skin ointment of the present disclosure. Specifically, as can be seen from the image on the top of FIG. 6, before the skin ointment for treating labia minora skin according to an embodiment of the present disclosure was applied, the vulva was severely swollen and showed symptoms accompanied by bleeding. In contrast, after the skin ointment for treating labia minora skin according to an embodiment of the present disclosure was periodically applied, as can be seen from (a) to (c) of FIG. 6, it can be confirmed that the bleeding of the vulva stopped and the original color of the vulva was restored. From FIG. 6, it can also be confirmed that, even in the case where no therapeutic effect is obtained through existing treatments such as hormone therapy, a significant effect of treating labia minora skin diseases such as vulvar vestibulitis can be obtained using the skin ointment for treating labia minora skin according to an embodiment of the present disclosure.

[symbol]

According to a skin ointment for treating labia minora skin according to the present disclosure, the skin ointment includes multi-growth factors as active ingredients, and is periodically applied to the vulva of a user so that the growth factors induce the regeneration of skin cells of the user, thereby increasing the effect of treating vulvar vestibulitis or wounds due to labia minora surgery, and addressing the fundamental causes thereof. In addition, according to the present disclosure, the skin ointment includes high concentrations of multi-growth factors and is also prepared as an ointment formulation, and thus is easily applied to the vulva of a user, and which, by excluding sex hormones, environmental hormones, steroid hormones, and antibiotic ingredients, does not break the hormonal balance of the user and can be used safely by pregnant or breast-feeding women.

[symbol]

The present disclosure described above can be modified or applied in various ways by those of ordinary skill in the art to which the present disclosure pertains, and the scope of the technical spirit of the present disclosure should be defined by the appended claimed.

1. A skin ointment for treating labia minora skin, the skin ointment comprising multi-growth factors as active ingredients,
wherein the multi-growth factors are multi-growth factors comprising FGF, bFGF, GM-CSF, HGF, IGF-1, IL-6,7,8, KGF, Matrix proteins, PDGF-AA, TGF-B2, TGF-B3, EGF, VEGF, and TNF, and
the skin ointment treating wounds due to labia minora surgery, and being in an ointment formulation, the ointment formulation being a semi-solid drug for external use.

2. The skin ointment of claim 1, wherein the skin ointment is periodically applied to a vulva of a user to treat vulvar vestibulitis.

3. A skin ointment for treating labia minora skin, the skin ointment comprising multi-growth factors as active ingredients,
wherein the multi-growth factors are multi-growth factors comprising FGF, bFGF, GM-CSF, HGF, IGF-1, IL-6,7,8, KGF, Matrix proteins, PDGF-AA, TGF-B2, TGF-B3, EGF, VEGF, and TNF, and
the skin ointment comprising, conditioned medium of growth factor containing the multi-growth factors with on a weight basis, 20%-40% of a high-concentration, and being in an ointment formulation, the ointment formulation being a semi-solid drug for external use.

4. The skin ointment of claim 3, wherein the high-concentration growth factor conditioned medium is included at a concentration of 300,000 ppm on a weight basis.

5. The skin ointment of claim 3, wherein the skin ointment is prepared as an ointment formulation comprising purified water, glycerin, Simmondsia chinensis (jojoba) seed oil, xanthan gum, a hydroxyethyl acrylate/sodium acryloyldimethyl taurate copolymer, squalane, polysorbate 60, Rosmarinus officinalis (rosemary) leaf oil, lactic acid, chlorphenesin, and sorbitan isostearate.

6. The skin ointment of claim 3, wherein the skin ointment excludes female hormones, environmental hormones, steroid hormones, and antibiotic ingredients.

7. The skin ointment of claim 1, wherein the skin ointment is prepared as an ointment formulation comprising purified water, glycerin, Simmondsia chinensis (jojoba) seed oil, xanthan gum, a hydroxyethyl acrylate/sodium acryloyldimethyl taurate copolymer, squalane, polysorbate 60, Rosmarinus officinalis (rosemary) leaf oil, lactic acid, chlorphenesin, and sorbitan isostearate.

8. The skin ointment of claim 1, wherein the skin ointment excludes female hormones, environmental hormones, steroid hormones, and antibiotic ingredients.
